(19) United States
(12) Patent Application Publication
(10) Pub. No.: US 2025/0136985 A1
(43) Pub. Date: May 1, 2025

(54) miR-141-3p MODULATORS AND USES THEREOF

(71) Applicant: University of Connecticut, Farmington, CT (US)

(72) Inventors: Rajkumar Verma, Woodbridge, CT (US); Sanjeev Kumar Yadav, Farmington, CT (US); Raman Bahal, Glastonbury, CT (US); Karishma Dhuri, Mansfield Center, CT (US)

(21) Appl. No.: 18/911,787

(22) Filed: Oct. 10, 2024

Related U.S. Application Data
(60) Provisional application No. 63/589,089, filed on Oct. 10, 2023.

(51) Int. Cl. C12N 15/113; A61K 9/00; A61K 9/50; A61K 47/10; A61P 7/00

(52) U.S. Cl. CPC C12N 15/113; A61K 9/0019; A61K 9/5031; A61K 47/10; A61P 7/00; C12N 2310/113; C12N 2310/3181

(57) ABSTRACT

Disclosed herein is a pharmaceutically effective composition comprising at least one of a phosphorothioate (PS)-based anti-miR-141-3p oligonucleotide; a peptide nucleic acid (PNA)-based anti-miR-141-3p oligonucleotide; and/or a gamma-peptide nucleic acid (γ-PNA)-based anti-miR-141-3p oligonucleotide; where at least one of the foregoing oligonucleotides is encapsulated in a biocompatible nanoparticle.

### CROSS-REFERENCE TO RELATED APPLICATIONS

This disclosure claims priority to U.S. Patent Provisional Application No. 63/589,089 filed on Oct. 10, 2023, the entire contents of which are hereby incorporated in their entirety.

### GOVERNMENT SUPPORT

This invention was made with government support under NS114981 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Jan. 14, 2025, is named “UCT0358US” and is 2,353 bytes in size. The Sequence Listing does not go beyond the disclosure in the application as filed.

### FIELD OF THE DISCLOSURE

This disclosure relates to agents and methods for modulating miR-141-3p. In particular, the disclosure relates to the agents for targeting miR-141-3p to alleviate the damage caused by ischemic stroke.

### BACKGROUND

The role of miRNA therapy in treating strokes has garnered increasing research interest since the first miRNA expression profiling study in cerebral ischemia was conducted in 2008. To date, a handful of studies have demonstrated the synthesis and validation of stable, practical, and cost-effective modulators of these miRNAs for stroke treatment. Anti-microRNA (referred to herein as “anti-miR”) based therapeutics have progressed in the realm of cancer treatment, yet remain unexplored for stroke treatment. Recombinant tissue plasminogen activator (rTPA)—also called alteplase (Activase) or tenecteplase (TNKase) is the common standard treatment for ischemic stroke, which dissolve clots within the occluded vessels. An injection of TPA is usually given intravenously within the first four and half hours of stroke onset. Mechanical thrombectomy, which involves removal of clot from a blood vessel is another approved therapy for stroke treatment, which has been shown to improve outcomes in patients with large vessel occlusions by restoring blood flow to the brain and reducing the severity of long-term disability when performed within a specific time window after symptom onset. However, there is not a single neuroprotective treatment available.

There is therefore a need for novel agents and methods that can prevent and/or alleviate the damage caused by an ischemic stroke.

### SUMMARY

Disclosed herein is a pharmaceutically effective composition comprising at least one of a phosphorothioate (PS)-based anti-miR-141-3p oligonucleotide; a peptide nucleic acid (PNA)-based anti-miR-141-3p oligonucleotide; and/or a gamma-peptide nucleic acid (γ-PNA)-based anti-miR-141-3p oligonucleotide; where at least one of the foregoing oligonucleotides is encapsulated in a biocompatible nanoparticle.

Disclosed herein too is a method of using a pharmaceutical composition, the method comprising administering a pharmaceutically effective dose of the pharmaceutical composition to a patient; wherein the pharmaceutical composition comprises at least one of a phosphorothioate (PS)-based anti-miR-141-3p oligonucleotide; a peptide nucleic acid (PNA)-based anti-miR-141-3p oligonucleotide; or a gamma-peptide nucleic acid (γ-PNA)-based anti-miR-141-3p oligonucleotide; where at least one of the foregoing oligonucleotides is encapsulated in a biocompatible nanoparticle; and monitoring a symptom of the patient; and modulating the pharmaceutically effective dose in response to observed results from the monitoring.

Disclosed herein too is a method of manufacturing a pharmaceutical composition, the method comprising dispersing at least one of a phosphorothioate (PS)-based anti-miR-141-3p oligonucleotide; a peptide nucleic acid (PNA)-based anti-miR-141-3p oligonucleotide; or a gamma-peptide nucleic acid (γ-PNA)-based anti-miR-141-3p oligonucleotide in aqueous solution; emulsifying the aqueous solution containing the oligonucleotides into an organic solvent containing a nanoparticle precursor to form a water-in-oil (W/O) emulsion; emulsifying the water-in-oil emulsion into a water-in-oil-in-water; and drying the water-in-oil-in-water emulsion to form nanoparticles.

### BRIEF DESCRIPTION OF THE FIGURES

FIG. 1 depicts the chemical structure of PS-141, PNA-141 and Sγ-PNA. The oligomer sequence of PS-141, PNA-141 and Sγ-PNA141 and their respective scrambled variants are shown in this figure;

FIGS. 2A-2C depicts cumulative release profile data for PS-141, PNA-141 and Sγ-PNA-141 and their respective scramble controls;

FIGS. 3A and 3B are graphs showing he loading efficiency for active PS-141, PNA-141 and Sγ-PNA in nanoformulations; data are shown as mean±standard error mean (derived from scanning electron microscopy (SEM);

FIGS. 4A and 4B are bar graphs that show dose-dependent effects of Sγ-PNA-141 on MTT cell viability assay (FIG. 4A) and a lactate dehydrase cellular toxicity assay (FIG. 4B). Data suggest that nanoparticles with Sγ-PNA-141 are highly tolerable with no apparent toxicity across a dose range of 0.015-1.5 μg/ml. Data are shown as mean±standard deviation (SD);

FIG. 5 is a graph that depicts a relative miR-141-3p gene expression analysis in the RNA isolated from HEK293 cells after 24 hrs of exposure to different concentrations of PS-141, PNA-141, and Sγ-PNA-141 nanoparticles (NPs). Three individual experiments were performed using different passage HEK293 cells. Data are presented as mean±standard deviation (SD) (*p<0.05 vs. Scr-control);

FIG. 6 depicts A) miR-141-3p expression levels in plasma samples acquired from aged-matched (58-90 years) healthy control (n=11) and stroke subjects (n=11) from acute and chronic time points. Data are mean±S.D (*p<0.05 vs. healthy control). B) the representative images show the changes in the infarct injury after the Sγ-PNA-141 treatment in post-stroke mice. C) Quantitative analysis of infarct volume compared to scr-control (* p<0.05 vs. scr-control, T-test) after a single intraperitoneal injection (15 μg/kg b.wt. of Sγ-PNA-141) given 4 hrs after onset of MCAo (n=11 Scr-control & n=12 Sγ-PNA-141). D) Sγ-PNA-141 treatment reduced relative miR-141-3p gene expression as revealed by analysis of RNA isolated from the perilesional cortex of mice after ischemic stroke (n=6 mice per group) (* p<0.05 vs. Scr-control, T-test). E) Correlation analysis showed significant correlation between miR-141-3p expression and infarct volume after stroke. F) Sγ-PNA-141 treatment also improves the neurological deficit score (n=22 scr-control and n=27 Sγ-PNA-141) (* p<0.05 vs. Scr-control). Each dot represents an individual sample. Data are mean±SD.

FIG. 7 depicts A) Direct target genes like Tgfβ2, Tgfβr1, and Tgfβr2 were increased after Sγ-PNA-141 treatment. B) Sγ-PNA-141 also reduced proinflammatory mRNA like I1-1β, I1-6, and Tnfα and C) increased Bdnf (a direct target) and Igf-1 levels. D) Sγ-PNA-141 treatment also increased the expression of mRNA for Smad2 and Smad3, which are the downstream targets of TGFβ family proteins. Each dot represents an individual sample (n=6 mice per group). (* p<0.05 vs. Scr-control, T-test). E) Western blot analysis of TGFβR1, TGFβR2, and BDNF (28 kd pro-BDNF) further confirm Sγ-PNA-141 de-represses the effect of miR-141-3p on the translation of these proteins (n=3-4 mice per group). F) Immunofluorescence images show increased TGFβR2 expression, which corroborated our qPCR and Western blot data. The reduced IBA-1 expression in the Sγ-PNA-141-treated group, confirming that Sγ-PNA-141 reduced neuroinflammation after stroke. Bar 100 and 20 μM;

FIG. 8A is a graph that shows γ-PNA-141 treatment also improves long term behavioral recovery on sensorimotor task by reducing latency to fall on a Rota rod test;

FIG. 8B is a graph that shows reversed learning and memory deficit caused by stroke as shown by discriminating index using Novel object recognition task; data are mean±S.D (*P<0.05 vs Scr-control and #P<0.05 vs Scr-control); and

FIG. 9 depicts the effect of Sγ-PNA-141 treatment on long-term neuroprotection, survival, and atrophy; A) Representative cresyl-violet-stained coronal brain sections of Sγ-PNA-141-treated mice and scr-control mice after 30 days stroke recovery; B) Quantitative analysis show that Sγ-PNA-141-treated mice had significantly less tissue loss compared to scr-controls (n=7-8 mice per group); C) Effect of Sγ-PNA-141 on survival (n=40-45 mice per group) and D) neurological deficit (ND) score (n=9-12 mice per group) after 30 days of stroke onset (* p<0.05 vs. Scr-control, t-test). Data are Mean±SD. Each dot represents an individual sample.

### DETAILED DESCRIPTION

### Definitions

“Blocking translation” refers to the inhibition of the process by which messenger RNA (mRNA) is used as a template to synthesize proteins in a cell. Translation is a key step in gene expression, where the information encoded in the mRNA is translated into a specific sequence of amino acids to form a protein.

Inducing degradation refers to the process by which a molecule, such as a microRNA (miRNA), causes the breakdown or destruction of messenger RNA (mRNA), thereby preventing it from being used to make proteins.

miR-141-3p is a microRNA, which is a small, non-coding RNA molecule that plays a crucial role in regulating gene expression. It is part of the miR-200 family.

Anti-miR-141-3p refers to an antisense oligonucleotide or inhibitor specifically designed to target and bind to the microRNA miR-141-3p to block its activity. The inhibitor prevents miR-141-3p from binding to its target mRNA. Anti-miR-141-3p is also referred to as an anti-miR-141-3p inhibitor.

A poly-A tail is a long chain of adenine nucleotides (typically around 50 to 250) that is added to the 3′ end of a messenger RNA (mRNA) molecule during RNA processing in eukaryotic cells. A eukaryotic cell is a type of cell that has a membrane-bound nucleus and other membrane-bound organelles.

γ-PNA, γ-PNA or gamma-PNA are all used interchangeably. Gamma PNA is a type of synthetic analog of DNA and RNA, part of the broader class of Peptide Nucleic Acids (PNA). PNAs are artificial polymers that mimic the structure of nucleic acids but have a different backbone, which imparts unique chemical and biological properties. In gamma PNA, the polymer backbone is modified by adding a substitution at the gamma position of the PNA monomer. This modification improves the binding properties and stability of the PNA in biological environments.

### DETAILED DESCRIPTION

Disclosed herein are pharmaceutical compositions that comprise peptide nucleic acid-based anti-miRs-141-3p that may be used for stroke therapy, in particular, to treat victims of ischemic strokes (hereinafter stroke victims). In an embodiment, the peptide nucleic acid-based anti-miRs-141-3p are encapsulated in nanoparticles and are used in pharmaceutically effective amounts to treat stroke victims. In an embodiment, the pharmaceutical composition comprises phosphorothioate (PS)-based anti-miR-141-3p, peptide nucleic acid (PNA)-based anti-miR-141-3p and/or gamma-peptide nucleic acid (γ-PNA)-based anti-miR-141-3p oligonucleotides encapsulated in biologically compatible (biocompatible) nanoparticles. Any combination of the foregoing oligonucleotides encapsulated by biocompatible nanoparticles may be delivered in a therapeutically effective dose to alleviate the after-effects of ischemic strokes.

In an embodiment, the pharmaceutical composition comprises phosphorothioate (PS)-based anti-miR-141-3p encapsulated in biologically compatible (biocompatible) nanoparticles. In an embodiment, the pharmaceutical composition comprises peptide nucleic acid (PNA)-based anti-miR-141-3p encapsulated in biologically compatible (biocompatible) nanoparticles. In an embodiment, the pharmaceutical composition comprises gamma-peptide nucleic acid (γ-PNA)-based anti-miR-141-3p oligonucleotides encapsulated in biologically compatible (biocompatible) nanoparticles. In an embodiment, the pharmaceutical composition comprises phosphorothioate (PS)-based anti-miR-141-3p, peptide nucleic acid (PNA)-based anti-miR-141-3p and gamma-peptide nucleic acid (γ-PNA)-based anti-miR-141-3p oligonucleotides encapsulated in biologically compatible (biocompatible) nanoparticles. In an embodiment, the γ-PNA-based anti-miR-141-3p modulator is a serine γ-PNA of SEQ01 with the sequence KCCATCTTTACCAGACAGTGTTAK.

In an embodiment, the biocompatible nanoparticles are preferably poly(lactic-co-glycolic acid) (PLGA) nanoparticles.

The phosphorothioate (PS)-based anti-miR-141-3p, peptide nucleic acid (PNA)-based anti-miR-141-3p and/or gamma-peptide nucleic acid (γ-PNA)-based anti-miR-141-3p oligonucleotides are sometimes referred to herein by their full names and at other times referred to as oligonucleotides. The term oligonucleotide encompasses one or more oligonucleotides, derivatives thereof, one or more analogues of the oligonucleotides, one or more pharmaceutically acceptable salts of the oligonucleotides, or a combination thereof. These compositions when delivered in pharmaceutically/therapeutically effective doses have been found to alleviate both acute and long-term deficits caused by ischemic strokes.

Disclosed herein too is a process for manufacturing the pharmaceutical compositions that comprise peptide nucleic acid-based anti-miRs-141-3p encapsulated in the nanoparticles. In particular, disclosed herein too is a process for manufacturing pharmaceutical compositions that comprises phosphorothioate (PS)-based anti-miR-141-3p, peptide nucleic acid (PNA)-based anti-miR-141-3p and/or gamma-peptide nucleic acid (γ-PNA)-based anti-miR-141-3p oligonucleotides encapsulated in biocompatible nanoparticles. The process comprises a single emulsion-solvent evaporation method or a double emulsion-solvent evaporation method (which is discussed in detail later). In a preferred embodiment, the process for manufacturing the nanoparticles comprises a double emulsion-solvent evaporation method.

miR-141-3p is produced as part of a precursor molecule called pri-miRNA, which is processed in the nucleus to form a shorter precursor miRNA (pre-miRNA). This pre-miRNA is exported to the cytoplasm, where it is further processed by the enzyme Dicer into a mature miRNA duplex. One strand of this duplex becomes the functional miRNA, in this case, miR-141-3p. The mature miR-141-3p is incorporated into a protein complex called the RNA-induced silencing complex (RISC). The Argonaute protein, a key component of RISC, helps guide miR-141-3p to its target mRNA. miR-141-3p interacts with its target mRNA by base-pairing with complementary sequences, usually found in the 3′ untranslated region (3′ UTR) of the mRNA. The binding is imperfect, but the “seed region” (nucleotides 2-8 of the miRNA) must have a substantial complementarity with the target sequence in the mRNA. Once miR-141-3p binds to the mRNA, it can inhibit gene expression in two main ways a) translational repression: miR-141-3p can prevent the mRNA from being translated into protein by interfering with the assembly of the ribosome or other factors needed for translation; or b) mRNA degradation: in some cases, miR-141-3p binding to mRNA leads to deadenylation (removal of the poly-A tail) and subsequent degradation of the mRNA. This reduces the amount of mRNA available for translation, effectively reducing protein levels. The ultimate effect of miR-141-3p interaction with mRNA is to decrease the expression of the target gene, either by reducing protein synthesis or by degrading the mRNA altogether.

When miR-141-3p inhibitors interact with miR-141-3p, they block the function of the microRNA, preventing it from binding to its target mRNA. As a result, the normal repression or degradation of the target mRNA by miR-141-3p is lifted. Without the miR-141-3p binding, the target mRNA is no longer subject to translational repression. This allows the ribosome and other translation machinery to assemble on the mRNA, leading to increased translation of the mRNA into its corresponding protein. In a similar manner, when the miR-141-3p inhibitor blocks miR-141-3p, the target mRNA is no longer degraded and becomes more stable. As a result, the mRNA levels may increase, leading to more available templates for translation.

In summary, when miR-141-3p inhibitors prevent the interaction between miR-141-3p and its target mRNA, the expression of the mRNA is typically restored. This means the mRNA can now be translated into protein, and the overall mRNA stability may improve, resulting in an increase in the protein levels that were previously downregulated by miR-141-3p.

As noted above, an agent for alleviating the effect of ischemic strokes includes at least one of a PS, a PNA, and/or a gamma peptide nucleic acid (PNA) based anti-miR-141-3p modulator. In an embodiment, the agent for alleviating the effect of ischemic strokes includes a PS based anti-miR-141-3p modulator. In an embodiment, the agent for alleviating the effect of ischemic strokes includes a PNA based anti-miR-141-3p modulator. In an embodiment, the agent for alleviating the effect of ischemic strokes includes a gamma peptide nucleic acid (γ-PNA) based anti-miR-141-3p modulator. In an embodiment, the agent for alleviating the effect of ischemic strokes includes a PS, a PNA, and a gamma peptide nucleic acid (γ-PNA) based anti-miR-141-3p modulator. In an embodiment, the γ-PNA-based anti-miR-141-3p modulator is a serine γ-PNA of SEQ01 with the sequence KCCATCTTTACCAGACAGTGTTAK.

In an embodiment, the anti-miR-141-3p modulator is an anti-miR-141-3p inhibitor—that directly binds to miR-141-3p and blocks its activity. The inhibitor prevents miR-141-3p from binding to its target mRNA, thereby reducing its gene-silencing effects. In another aspect, disclosed is a composition including a PS, a PNA, and/or a gamma peptide nucleic acid (γ-PNA) based anti-miR-141-3p modulator (for example, anti-miR-141-3p inhibitor) encapsulated within poly(lactide-co-glycolide) (PLGA)-based nanoparticles (NPs).

In an embodiment, the composition includes a phosphorothioate (PS)-based anti-miR-141-3p modulator (for example, anti-miR-141-3p inhibitor) encapsulated within poly(lactide-co-glycolide) (PLGA)-based nanoparticles (NPs). In an embodiment, the composition includes a PNA-based anti-miR-141-3p modulator (for example, anti-miR-141-3p inhibitor) encapsulated within poly(lactide-co-glycolide) (PLGA)-based nanoparticles (NPs). In an embodiment, the composition includes a gamma peptide nucleic acid (γ-PNA)-based anti-miR-141-3p modulator (for example, anti-miR-141-3p inhibitor) encapsulated within poly(lactide-co-glycolide) (PLGA)-based nanoparticles (NPs). In an embodiment, the composition includes a PS, a PNA, and a gamma peptide nucleic acid (γ-PNA) based anti-miR-141-3p modulator (for example, anti-miR-141-3p inhibitor) encapsulated within poly(lactide-co-glycolide) (PLGA)-based nanoparticles (NPs). In an embodiment, the γ-PNA-based anti-miR-141-3p oligonucleotide is a serine γ-PNA with a DNA or RNA sequence. In another embodiment, the γ-PNA-based anti-miR-141-3p oligonucleotide is a serine γ-PNA of SEQ01 with the sequence KCCATCTTTACCAGACAGTGTTAK.

In an embodiment, the anti-miR-141-3p modulator (which functions primarily as an anti-miR-141-3p inhibitor) comprises gamma-PNA (γ-PNA). In an embodiment, the gamma-PNA (γ-PNA) is a serine γ-PNA of SEQ01 with the sequence KCCATCTTTACCAGACAGTGTTAK. Gamma PNA (γ-PNA) refers to a modified version of peptide nucleic acid (PNA), where the backbone of the PNA molecule has been altered by introducing modifications at the gamma position of the PNA monomer. This modification often involves the addition of chemical groups to the gamma carbon atom, which influences the hydrophilicity, flexibility, stability, and binding properties of the PNA molecule. Gamma-PNA often shows improved binding affinity to complementary nucleic acid strands (DNA or RNA) compared to unmodified PNA. The gamma modification often makes the backbone more rigid, which helps the PNA form more stable duplexes with the target DNA or RNA.

In an embodiment, the gamma-PNA used in the miR-141-3p inhibitor comprises a peptide nucleic acid in which the gamma position of the nucleic acid is altered to introduce a hydrophilic (water-attracting) group into the backbone of the nucleic acid. Examples of nucleic acid thus altered include serine-gamma-PNA, ethylene glycol-gamma-PNA, or the like. In a serine-gamma-PNA, the gamma position of the PNA monomer, which is part of the backbone structure, is modified with a serine residue. The structure of serine-gamma-PNA is shown below in formula (1):

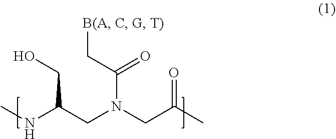

Serine is an amino acid with a hydroxyl (—OH) group, which can influence the structural and chemical properties of the molecule. This modification at the gamma position is useful because it increases the overall hydrophilicity (water affinity) and can impact how the PNA interacts with target nucleic acids. Ethylene glycol gamma nucleic acid (EG-γNA) is another type of gamma-modified nucleic acid analog in which the backbone contains ethylene glycol units at the gamma position of the peptide nucleic acid (PNA) backbone.

In an embodiment, the gamma-PNA used in the miR-141-3p inhibitor may be further subjected to a phosphorothioate (PS) modification. This modification refers to the replacement of a non-bridging oxygen atom in the phosphate backbone of nucleic acids (like DNA or RNA) with a sulfur atom. A phosphorothioate (PS)-based anti-miR-141-3p comprises a synthetic oligonucleotide sequence designed to complement miR-141-3p, where the backbone phosphate groups are modified by replacing one of the non-bridging oxygen atoms with a sulfur atom, providing increased nuclease resistance compared to natural RNA; essentially, it's a modified version of the miR-141-3p sequence with a sulfur-containing backbone, allowing it to bind to and inhibit the activity of the target miRNA molecule.

A peptide nucleic acid (PNA)-based anti-miR-141-3p comprises a synthetic, neutral backbone composed of repeating N-(2-aminoethyl) glycine units, with the natural nucleobases (adenine, cytosine, guanine, and thymine) attached via a methylene carbonyl linker, specifically designed to be complementary to the sequence of miR-141-3p, allowing it to bind and inhibit the microRNA's function. This structure provides high binding affinity to the target miRNA while being resistant to degradation by nucleases.

A γ-PNA-based anti-miR-141-3p comprises a synthetic peptide nucleic acid (PNA) backbone with a “gamma” modification, meaning a side chain attached at the gamma position of the amino acid backbone, where the nucleobases complementary to miR-141-3p are attached, allowing it to specifically bind and inhibit the activity of this microRNA. It is a modified PNA designed to target and neutralize miR-141-3p by base pairing.

As noted above, the phosphorothioate (PS)-based anti-miR-141-3p, peptide nucleic acid (PNA)-based anti-miR-141-3p and/or gamma-peptide nucleic acid (γ-PNA)-based anti-miR-141-3p oligonucleotides are encapsulated in biocompatible nanoparticles. The biocompatible nanoparticles are preferably compatible with body fluids and can either undergo swelling or dissolution in body fluids to release the pharmaceutical cargo (e.g., the phosphorothioate (PS)-based anti-miR-141-3p, the peptide nucleic acid (PNA)-based anti-miR-141-3p and/or the gamma-peptide nucleic acid (γ-PNA)-based anti-miR-141-3p oligonucleotides). It is desirable for the nanoparticles to be compatible with the oligonucleotides that they are required to transport into the body.

The biocompatible nanoparticles can include poly(lactic-co-glycolic acid) (PLGA) nanoparticles, lipid nanoparticles, chitosan nanoparticles, silica nanoparticles, silica nanoparticles, polycaprolactone nanoparticles, dendrimer nanoparticles, gold nanoparticles, polylactic nanoparticles, albumin nanoparticles, calcium phosphate nanoparticles, or a combination thereof. PLGA nanoparticles are preferred.

PLGA nanoparticles are synthesized using a variety of techniques, depending on the desired properties of the nanoparticles, such as size, drug encapsulation efficiency, and release profile. PLGA nanoparticles are used for drug delivery, due to their biocompatibility, biodegradability, and ability to control drug release.

PLGA nanoparticles are manufactured using a single emulsion-solvent evaporation method or a double emulsion-solvent evaporation method. Either of the foregoing methods may be used to encapsulate the phosphorothioate (PS)-based anti-miR-141-3p, peptide nucleic acid (PNA)-based anti-miR-141-3p and/or gamma-peptide nucleic acid (γ-PNA)-based anti-miR-141-3p oligonucleotides in the PLGA nanoparticles.

In the single emulsion-solvent evaporation method, PLGA along with one or more of the phosphorothioate (PS)-based anti-miR-141-3p, peptide nucleic acid (PNA)-based anti-miR-141-3p and/or gamma-peptide nucleic acid (γ-PNA)-based anti-miR-141-3p oligonucleotides are dissolved in a suitable organic solvent. Acid-terminated and ester-terminated PLGA's may be used in the manufacture of the nanoparticles. The organic phase containing PLGA and one or more of the foregoing oligonucleotides is emulsified in an aqueous solution containing a surfactant (e.g., polyvinyl alcohol (PVA)) under high-speed stirring or sonication. This forms an oil-in-water (O/W) emulsion. In an embodiment, the organic solvent may be evaporated under reduced pressure or through stirring, leading to the hardening of the PLGA into solid nanoparticles.

The nanoparticles comprising the PLGA and one or more of the foregoing oligonucleotides are collected by centrifugation or filtration, and then washed with water to remove any remaining surfactant or free drug. The nanoparticles are typically freeze-dried or air-dried for long-term storage.

In the double emulsion-solvent evaporation method, the aqueous solution containing one or more of the foregoing oligonucleotides is emulsified into an organic solvent containing PLGA (e.g., ethyl acetate) to form a water-in-oil (W/O) emulsion. This step is usually performed with high-speed homogenization or sonication. The W/O emulsion is then further emulsified into an aqueous phase containing a surfactant (e.g., PVA), forming a water-in-oil-in-water (W/O/W) double emulsion. The organic solvent is evaporated, causing the PLGA to harden and form nanoparticles. The nanoparticles are collected via centrifugation or filtration and washed to remove any free drug or surfactant. The nanoparticles are freeze-dried or air-dried for storage.

In an embodiment, the double emulsion-solvent evaporation method is preferred because the oligonucleotides are hydrophilic. This method provides for control over particle size and drug loading.

In an embodiment, the nanoparticles have average particle sizes as determined by light scattering of about 10 to about 350 nanometers, preferably about 20 to about 100 nanometers. The average particle size is the hydrodynamic radius.

In an embodiment, the nanoparticles contain the PLGA in an amount of about 2 to about 99.5 weight percent (wt %), based on a total weight of the nanoparticles. In a preferred embodiment, the nanoparticles contain the PLGA in an amount of about 10 to about 98 weight percent, based on a total weight of the nanoparticles.

In an embodiment, the nanoparticles contain the foregoing nucleotides in an amount of about 0.01 to about 8 weight percent, based on a total weight of the nanoparticles. In a preferred embodiment, the nanoparticles contain the foregoing nucleotides in an amount of about 0.1 to about 5 wt %, based on a total weight of the nanoparticles.

In one embodiment, in one method of using the composition, the nanoparticles may be dispersed in a suitable carrier liquid and administered to a patient orally, intravenously, transdermally, intraperitoneally topically and/or subcutaneously to alleviate the symptoms of an ischemic stroke. Suitable carrier liquids include water, pharmaceutically ingestible alcohols, saline, or a combination thereof. Pharmaceutically ingestible alcohols include ethanol, glycerol, propylene glycol, sorbitol, mannitol, polyethylene glycol, xylitol, erythritol, butylene glycol, or a combination thereof.

When administered topically, alcohols that are not necessarily ingestible may be used. Examples of such alcohols include isopropanol, benzyl alcohol, cetyl alcohol, lauryl alcohol, or a combination thereof.

In an embodiment, a method of using a pharmaceutical composition comprises administering a pharmaceutically effective dose of the pharmaceutical composition to a patient;

wherein the pharmaceutical composition comprises at least one of a phosphorothioate (PS)-based anti-miR-141-3p oligonucleotide; a peptide nucleic acid (PNA)-based anti-miR-141-3p oligonucleotide; and/or a gamma-peptide nucleic acid (γ-PNA)-based anti-miR-141-3p oligonucleotide; where at least one of the foregoing oligonucleotides is encapsulated in a biocompatible nanoparticle. The patients symptoms may be monitored following which the pharmaceutically effective dose may be varied (increased or decreased) in response to observed results from the monitoring.

In orally administered formulations, the nanoparticles may be present in an amount of about 0.01 to about 20 wt %, based on a total weight of the formulation. In intravenously administered formulations, the nanoparticles may be present in an amount of about 0.1 to about 1 wt %, based on a total weight of the formulation.

In intraperitoneally administered formulations, the nanoparticles may be present in an amount of about 0.01 to about 2 wt %, based on a total weight of the formulation.

In an embodiment, a pharmaceutically/therapeutically effective dose comprises about 0.4 to about 4 microgram per kilogram of human weight.

The pharmaceutical compositions and the formulations, the methods of manufacture thereof and the methods of use disclosed herein are exemplified by the following non-limiting examples.

### EXAMPLES

### Example 1

This example was conducted to demonstrate the manufacturing of the nanoparticles that contain both PLGA and the nucleotides. The PLGA nanoparticles containing anti-miR-141-3p or PNA was manufactured by using a double emulsion solvent evaporation technique. The PLGA is present in an acetone-dichloromethane (DCM) at a 2:1 volume ratio. This formed the oil phase. The anti-miR-141-3p or PNA was present in water. This formed the water phase. The water in oil (w/o) emulsion was formed by adding the water phase to the organic phase. The w/o emulsion was added to 5% polyvinyl alcohol (PVA) to form the w/o/w emulsion. The suspension was then added to 0.3 wt % PVA and stirred at room temperature overnight. The suspension was then lyophilized to obtain the PLGA nanoparticles containing PNA or anti-miR-141-3p.

### Example 2

This example was conducted to determine the efficacy of the nanoparticles in alleviating the effects of ischemic strokes.

The PS, PNA, and gamma-PNA anti-miR-141-3p oligonucleotides used in this example were encapsulated in PLGA nanoparticles (NPs) and were manufactured via a double emulsion solvent evaporation technique. Post physicochemical characterization, the in vitro safety and efficacy of these inhibitors were assessed in HEK293 cells employing MTT, LDH cytotoxicity assays, and qPCR for gene expression analysis. The in vivo efficacy studies utilized the most potent gamma-PNA based anti-miR-141-3p, administered intraperitoneally 4 hours post a 60-minute transient middle cerebral artery occlusion (MCAO). The inhibitors were tested on HEK293 cells (a common human cell line) to see if they were safe and effective. Two methods were used to evaluate how toxic the inhibitors are to the cells: a) an MTT assay: Measures cell viability based on the metabolic activity of living cells; and b) LDH cytotoxicity assay: Measures the release of lactate dehydrogenase (LDH) from damaged cells, indicating cell death or damage.

Quantitative PCR (qPCR) was used to analyze how these inhibitors affect the expression of specific genes. This is a technique used to measure changes in the levels of mRNA, indicating changes in gene activity. After testing in cells, the most potent inhibitor was selected for testing in a living organism. The chosen inhibitor was a gamma-PNA (peptide nucleic acid) based anti-miR-141-3p. This is a molecule designed to inhibit microRNA-141-3p, which could be involved in disease processes (e.g., stroke).

The inhibitor was injected into the peritoneal cavity (the area surrounding abdominal organs) of the animal model. This is a model for inducing a stroke by temporarily blocking blood flow to part of the brain (the middle cerebral artery). The inhibitor was administered 4 hours after a 60-minute transient MCAO, meaning the blood flow was blocked for 60 minutes to induce a stroke, and then the inhibitor was given 4 hours after this event to study its potential protective effects.

Mice were sacrificed either 3 (acute) or 30 (chronic) days post-stroke. Brain tissues from the acute cohort were analyzed for miRNA and target mRNA levels, along with infarct volume assessment. The chronic cohort mice underwent weekly behavioral tasks to evaluate sensorimotor deficits or recovery.

The HEK-293 cells, exposed for twenty-four hours, exhibited no mortality or toxicity within a concentration range of 0.015-1.5 micrograms per milliliter (μg/mL) for various anti-miR-141-3p compounds. The gamma-PNA based anti-miR demonstrated a significantly higher efficacy in inhibiting miR-141-3p (IC50-0.05 μg/mL) when compared to PS (IC50>1.5 μg/mL) or regular PNA (IC50>1.5 μg/mL). A single dose of gamma-PNA-based anti-miR significantly reduced infarct injury (P<0.05 vs. Scramble control) and brain tissue miR-141-3p levels (>2-fold vs. scramble control). The treatment with gamma-PNA also facilitated a rapid improvement in sensorimotor deficits as evidenced in the rotarod task, and aided recovery from learning and memory deficits post-stroke. The NPs of gamma-PNA-based anti-miR-141-3p were proven to be safe and potent both in vitro and in vivo. They effectively alleviated infarct injury and improved neurobehavioral deficits, showcasing promising translational potential for ischemic stroke therapy.

In an embodiment, the nanoparticles delivered peptide nucleic acid-based anti-miRs-141-3p for potential stroke therapy. In certain embodiments, three different classes of miRNA inhibitor, i.e., PS, PNA, and gammaPNA-based anti-miR-141-3p for stroke therapy. For delivery, FDA-approved PLGA NP formulations were utilized. It was observed that the PLGA formulations could encapsulate optimal amounts of PS, PNA, and gamma PNA-based anti-miR-141-3p, thereby generating NPs with superior physicochemical features; uniform spherical morphology, size, distribution, and surface charge density. Additionally, the release profile of NPs using UV-vis-based techniques were studied and examined the significant uptake of NPs containing fluorophore-tagged anti-miR. It was also established that systemic delivery of these NPs reduced miR-141-3p levels, decreased the infarct volume, and improved behavioral deficits post-stroke.

### Example 3

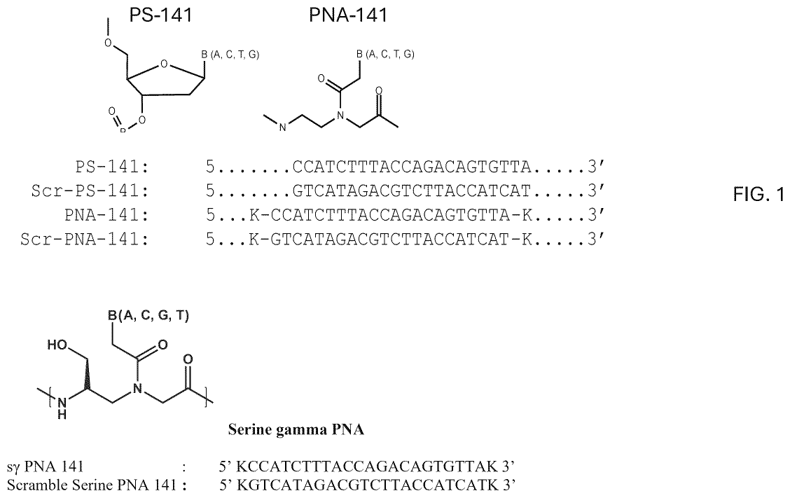

A prior study in C57BL/6 mice showed persistent elevated miR-141-3p levels for two weeks after stroke. miR-141-3p inhibition reduced mortality by 20% in post-stroke isolated mice. In this example, anti-miR-serine PNA sequences complementary to target miR-141-3p were manufactured using Boc-based solid-phase synthesis. These are shown as Sγ-PNA-141 in the FIG. 1. FIG. 1 depicts the chemical structure of PS-141, PNA-141 and Sγ-PNA. The oligomer sequence of PS-141, PNA-141 and Sγ-PNA141 and their respective scrambled variants are shown in this figure

Further, quality control assessment of PNAs was performed using reverse-phase HPLC and mass spectrometry analysis. Scrambled PNA oligomers were used as controls (Scr-Sγ-PNA-141). These are also depicted in the FIG. 1.

### Example 4

In prior studies, it was determined that acid terminated PLGA and ester terminated PLGA NPs (containing equal ratios of poly-lactic acid and poly-glycolic acid, 50:50) can effectively deliver PS and PNA-based anti-miRs to the affected site in a patient. In this study, NP formulations were developed that can encapsulate and deliver the optimum amount of serine PNA based anti-miR 141 and their scrambled (Scr) controls.

Table 1 below depicts characterization of PLGA NPs containing antimiR-141 sequences. Mean diameter and polydispersity index (PDI) was calculated using dynamic light scattering (DLS), and the surface charge was calculated using zeta potential.

TABLE 1

| | | | Zeta Potential |
|---|---|---|---|
| Nanoformulation | DLS (nm ± SEM) | PDI (±SEM) | (mV ± SEM) |
| Sγ-PNA-141 | 158.5 ± 6.7 | 0.12 ± 0.071 | −19.07 ± 1.42 |
| Scr-Sγ-PNA-141 | 146.8 ± 4.6 | 0.16 ± 0.053 | −15.93 ± 3.73 |

### Example 5

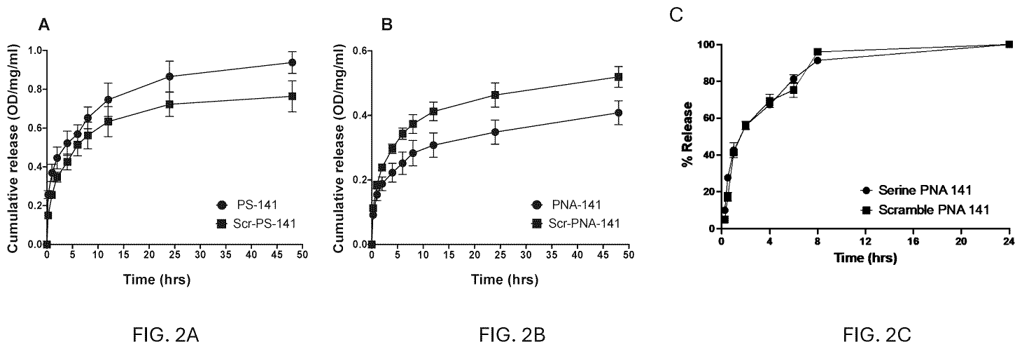

In this example, the nucleic acid release profile from PLGA NPs was studied by re-suspending NPs in the phosphate buffer saline followed by determining their UV-Vis absorbance at 260 nm at indicated time points as in FIG. 2. FIG. 2 is a graph that shows cumulative release profile data of (Sγ-PNA-141 and Scr-gPNA-141). Data are shown as mean±standard error mean (obtained from scanning electron microscopy (SEM)). Results indicate that the NPs showed maximum release at 24 hours. These results suggested that NPs containing a PNA-based anti-miRs, and anti-miRs were released from the NP formulation without any hindrance from the PLGA polymer.

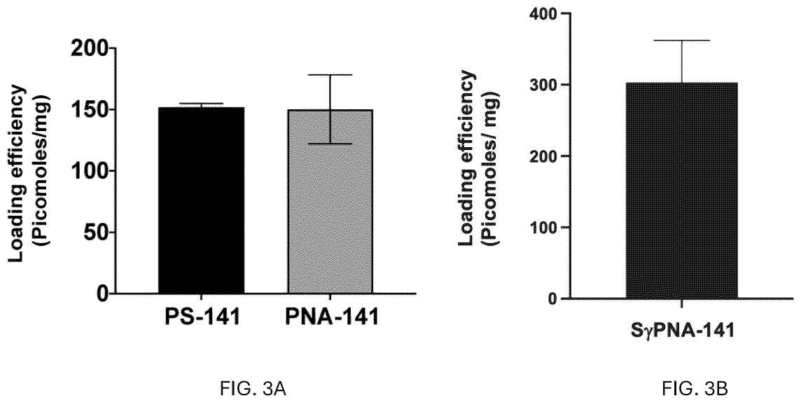

FIG. 3 is a graph that shows Sγ-PNA loading analysis. Data are shown as mean±standard error mean (SEM). An average loading of ˜170 picomoles/mg was observed for Sγ-PNA-141 in PLGA NPs (FIG. 3). The loading results imply that Sγ-PNA anti-miRs can be effectively encapsulated in PLGA NPs by double emulsion solvent evaporation technique.

### Example 6

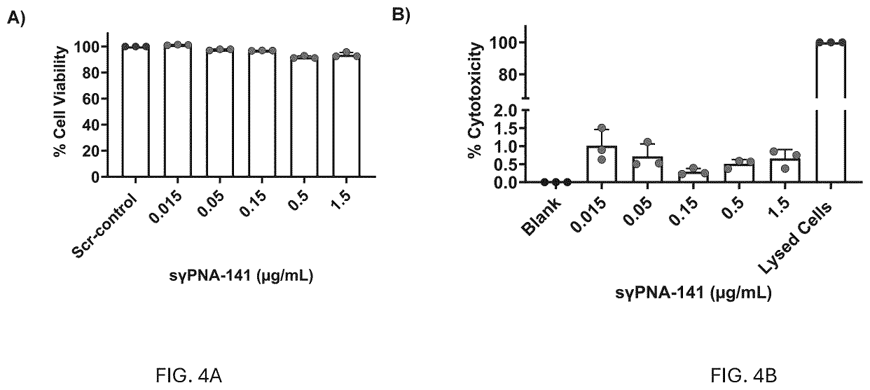

In this example, the safety of Sγ-PNA-141 NPs (γ PNA-141 in short) was evaluated in HEK cells. HEK293 cells were exposed to the serine PNA-141 NPs at a concentration of 0.015-1.5 μg/ml and the cell viability was measured by MTT assay. Cytotoxicity was not observed for Sγ-PNA-141 NPs at all the evaluated doses (see FIG. 4). This suggests that the NPs are non-toxic and well tolerated even at higher dose. FIG. 4A depicts dose dependent effect of Sγ-PNA 141 NP on MTT cell viability assay, and FIG. 4B depicts lactate dehydrase cellular toxicity assay; the data suggest that NPs pf gamma PNA anti-miR 141-3p are highly safe with no toxicity at dose range of 0.015-1.5 μg/ml concentrations. Data are shown as mean±standard deviation (SD).

### Example 7

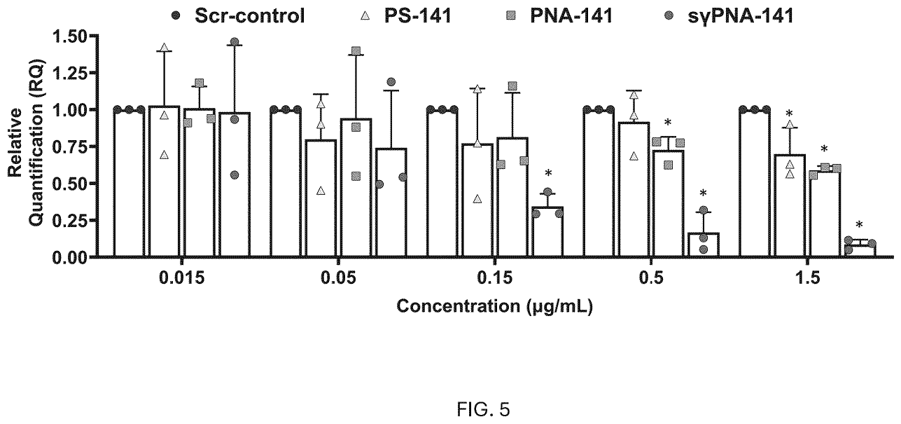

To compare the efficacy of Sγ-PNA-anti-miR 141 NPs for miR-141 inhibition, PS and regular PNA NPs were used in addition to the scramble control. The HEK293 cells were exposed to the PS, regular PNA, and serine PNA NPs at the conc. 0.015-1.5 ug/mL. After 24 hrs, total RNA (including miRNAs) was isolated from the cells and the expression of miR-141 was measured by Real time PCR. The results indicated that Sγ-PNA based anti-miR 141 show higher efficacy in inhibiting miR-141 compared to PS and regular PNA NPs in in vitro HEK cell based model. FIG. 5 is a graph that depicts a relative miR-141-3p gene expression analysis in the RNA isolated from HEK293 cells after 24 hrs of exposure to different concentrations of PS-141, PNA-141, and Sγ-PNA-141 nanoparticles (NPs). Three individual experiments were performed using different passage HEK293 cells. Data are presented as mean±standard deviation (SD) (*p<0.05 vs. Scr-control).

### Example 8

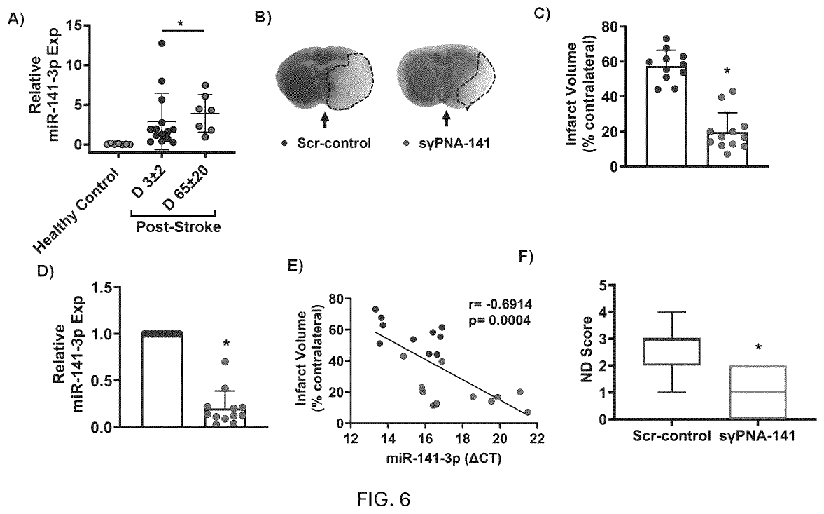

This example is conducted to validate in-vivo efficacy using a stroke mouse model. First it was determined whether this miRNA is expressed in human control and stroke subjects in order to test miR-141-3p as being a good therapeutic target in human beings. We wanted to determine if miR-141-3p would be useful as an inhibitor to treat strokes (FIG. 6A). We found that after a stroke, miR-141-3p levels increase.

The efficacy of NPs of Sγ-PNA based miR-141-3p inhibitors (γ-PNA 141) and scramble control was evaluated using mouse model of ischemic stroke. Systemic delivery of these NPs by intraparetoneal administration 4 hour after stroke onset was performed and mice were sacrificed after 3 days of stroke. qPCR analysis of miR-141-3p from the total RNA isolated from the ipsilateral cortex of both groups was performed. It was found that γ-PNA 141 reduced the miR-141-3p gene expression by an a amount greater than 3 fold as compared to scrambled control. Our results indicated the high efficacy of γ-PNA 141 in inhibiting miR-141-3p levels in the brain as shown in the FIG. 6B-D and we also showed a significant correlation between miR-141-3p expression and infarct volume after stroke in FIG. 6E and improvement on neurological deficit (see FIG. 6F).

FIG. 6 depicts A) miR-141-3p expression levels in plasma samples acquired from aged-matched (58-90 years) healthy control (n=11) and stroke subjects (n=11) from acute and chronic time points. Data are mean±S.D (*p<0.05 vs. healthy control). B) the representative images show the changes in the infarct injury after the Sγ-PNA-141 treatment in post-stroke mice. C) Quantitative analysis of infarct volume compared to scr-control (* p<0.05 vs. scr-control, T-test) after a single intraperitoneal injection (15 μg/kg b.wt. of Sγ-PNA-141) given 4 hrs after onset of MCAo (n=11 Scr-control & n=12 Sγ-PNA-141). D) Sγ-PNA-141 treatment reduced relative miR-141-3p gene expression as revealed by analysis of RNA isolated from the perilesional cortex of mice after ischemic stroke (n=6 mice per group) (* p<0.05 vs. Scr-control, T-test). E) Correlation analysis showed significant correlation between miR-141-3p expression and infarct volume after stroke. F) Sγ-PNA-141 treatment also improves the neurological deficit score (n=22 scr-control and n=27 Sγ-PNA-141) (* p<0.05 vs. Scr-control). Each dot represents an individual sample. Data are mean±standard deviation (SD).

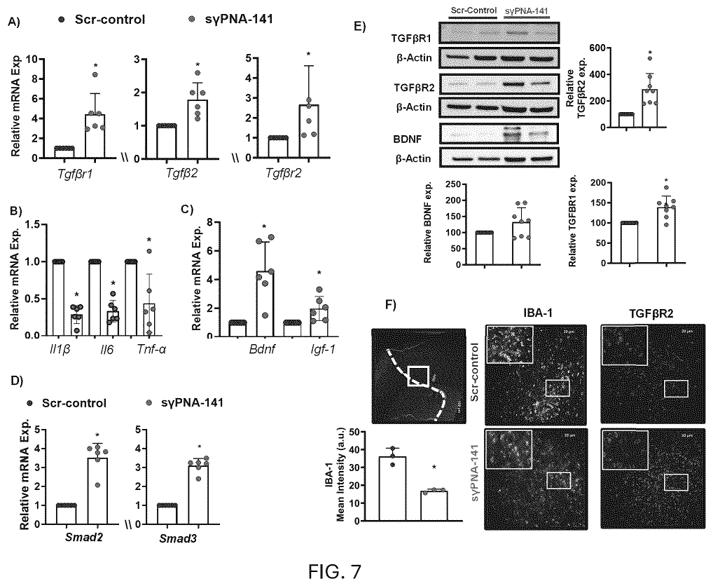

FIG. 7 includes data that depicts A) Direct target genes like Tgfβ2, Tgfβr1, and Tgfβr2 were increased after Sγ-PNA-141 treatment. B) Sγ-PNA-141 also reduced proinflammatory mRNA like I1-1β, Ia-6, and Tnfα and C) increased Bdnf (a direct target) and Igf-1 levels. D) Sγ-PNA-141 treatment also increased the expression of mRNA for Smad2 and Smad3, which are the downstream targets of TGFβ family proteins. Each dot represents an individual sample (n=6 mice per group). (* p<0.05 vs. Scr-control, T-test). E) Western blot analysis of TGFβR1, TGFβR2, and BDNF (28 kd pro-BDNF) further confirm Sγ-PNA-141 de-represses the effect of miR-141-3p on the translation of these proteins (n=3-4 mice per group). F) Immunofluorescence images show increased TGFβR2 expression, which corroborated our qPCR and Western blot data. The reduced IBA-1 expression in the Sγ-PNA-141-treated group, confirming that Sγ-PNA-141 reduced neuroinflammation after stroke. Bar 100 and 20 μM.

### Example 9

This example was conducted to determine neuroprotective effects in-vivo. To measure the effect of γ-PNA 141 treatment on infarct injury, triphenyl tetrazolium chloride (TTC) staining technique was used to measure the viability of the tissue. Dead tissue remains unstained and can be easily measured to determine infarct injury after stroke. It was determined that after a single intraperitoneal injection (15 μg/kg b.w 4 hrs after MCAo) of gamma PNA-141 containing NPs significantly (p<0.05 vs. Scr-control) reduced infarct volume at 3 days after stroke was observed (See FIG. 6.). FIG. 6B depicts micrographs showing that anti-miR-141 treatment reduce infarct injury. Left panel show representative images. NPs of gamma-PNA bases anti-miR 141-3p show significantly reduced infarct volume in post-stroke mice compared to Scr-control (* p<0.05 vs. Scr-control) after a single intraperitoneal injection (15 μg/kg b.w of γ PNA-141) given 4 hrs. after MCAo (right panel). The data is presented as Mean±S.D.

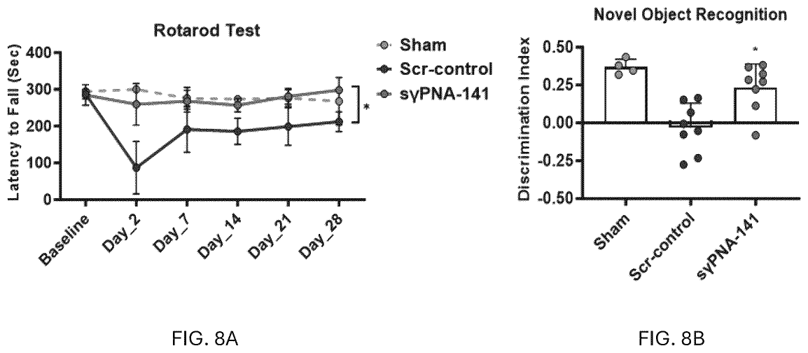

γ-PNA 141 treatment also improved sensorimotor performance (See FIG. 8A) as well as learning and memory deficit (see FIG. 8B) during chronic recovery after a stroke. FIG. 8A is a graph that shows γ-PNA-141 treatment also improves long term behavioral recovery on sensorimotor task by reducing latency to fall on a Rota rod test. FIG. 8B is a graph that shows reversed learning and memory deficit caused by stroke as shown by discriminating index using Novel object recognition task; data are mean±S.D (*P<0.05 vs scr-control and #P<0.05 vs scr-control).

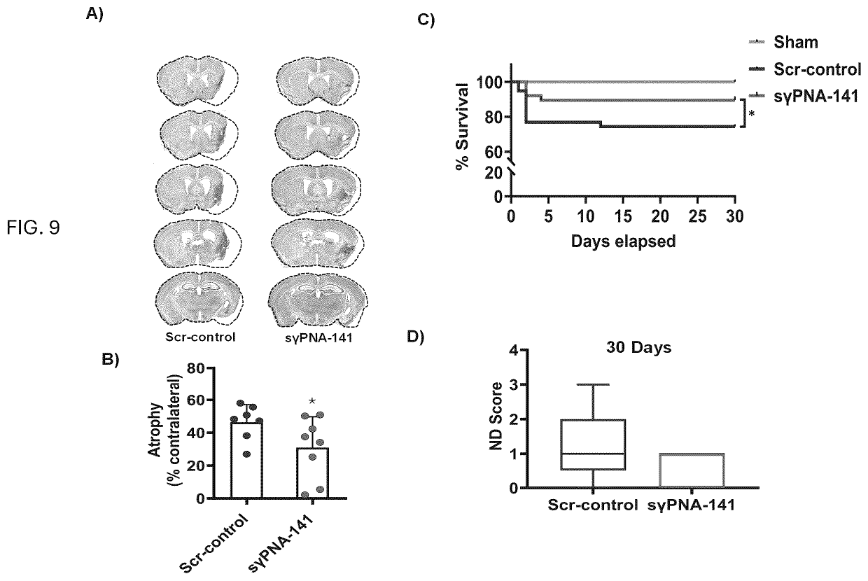

γ-PNA 141 treatment also reduced tissue loss measured 30 days after stroke (p=0.0357) (FIGS. 9A and 9B). Sγ-PNA-141-treated mice had a lower mortality rate (p=0.0349) compared to the scr-control-treated mice after stroke (FIG. 9C). effect on neurological deficit score between the treatment and control groups at the end of the chronic cohort (FIG. 9D). These data suggest that Sγ-PNA-141 can impart sustained neuroprotective effects during chronic recovery after stroke and rescue tissue loss in the penumbra after ischemic stroke.

FIG. 9 depicts the effect of Sγ-PNA-141 treatment on long-term neuroprotection, survival, and atrophy; A) Representative cresyl-violet-stained coronal brain sections of Sγ-PNA-141-treated mice and scr-control mice after 30 days stroke recovery; B) Quantitative analysis show that Sγ-PNA-141-treated mice had significantly less tissue loss compared to scr-controls (n=7-8 mice per group); C) Effect of Sγ-PNA-141 on survival (n=40-45 mice per group) and D) neurological deficit (ND) score (n=9-12 mice per group) after 30 days of stroke onset (* p<0.05 vs. Scr-control, t-test). Data are Mean±SD. Each dot represents an individual sample.

In summary, the pharmaceutically and therapeutically effective compositions disclosed herein are useful for reducing the acute and long term effects of strokes when delivered in pharmaceutically effective doses.

The high stability of the agents and compositions disclosed herein in blood circulation, the ability to optimize delivery to a target organ, the efficient inhibition of the target, and favorable disease outcomes such as reduction in stroke size post-treatment are some of the novel features of these pharmaceutical compositions. This novel therapy/method could be administered as an adjunctive medical treatment to clot busters and/or thrombectomy, and may also be given alone for post-stroke treatment.

Embodiments disclosed here are not limiting of the subject matter and is merely exemplary. Various other components may be included and called upon for providing for aspects of the teachings herein. For example, additional materials, combinations of materials and/or omission of materials may be used to provide for added embodiments that are within the scope of the teachings herein.

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising,” “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, “optional” or “optionally” means that the subsequently described event or circumstance may or may not occur, and that the description includes instances where said event or circumstance occurs and instances where it does not.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

A significant change is any detectable change that is statistically significant in a standard parametric test of statistical significance such as Student's t-test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112 (f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A pharmaceutically effective composition comprising:
at least one of a phosphorothioate (PS)-based anti-miR-141-3p oligonucleotide; a peptide nucleic acid (PNA)-based anti-miR-141-3p oligonucleotide; or a gamma-peptide nucleic acid (γ-PNA)-based anti-miR-141-3p oligonucleotide; where at least one of the foregoing oligonucleotides is encapsulated in a biocompatible nanoparticle.

2. The pharmaceutically effective composition of claim 1, comprising the gamma-peptide nucleic acid (γ-PNA)-based anti-miR-141-3p oligonucleotide encapsulated in the biocompatible nanoparticle.

3. The pharmaceutically effective composition of claim 1, wherein the biocompatible nanoparticles include poly(lactic-co-glycolic acid) nanoparticles, lipid nanoparticles, chitosan nanoparticles, silica nanoparticles, silica nanoparticles, polycaprolactone nanoparticles, dendrimer nanoparticles, gold nanoparticles, polylactic nanoparticles, albumin nanoparticles, calcium phosphate nanoparticles, or a combination thereof.

4. The pharmaceutically effective composition of claim 1, wherein the biocompatible nanoparticles include poly(lactic-co-glycolic acid) nanoparticles.

5. The pharmaceutically effective composition of claim 1, wherein the gamma-peptide nucleic acid (γ-PNA)-based anti-miR-141-3p oligonucleotide is altered with a hydrophilic group introduced into a backbone of a nucleic acid.

6. The pharmaceutically effective composition of claim 4, wherein the hydrophilic group is derived from serine, ethylene glycol, or a combination thereof.

7. The pharmaceutically effective composition of claim 1, wherein the γ-PNA-based anti-miR-141-3p oligonucleotide is a serine γ-PNA of SEQ01 with the sequence KCCATCTTTACCAGACAGTGTTAK.

8. The pharmaceutically effective composition of claim 1, wherein the phosphorothioate (PS)-based anti-miR-141-3p oligonucleotide; the peptide nucleic acid (PNA)-based anti-miR-141-3p oligonucleotide; and/or the gamma-peptide nucleic acid (γ-PNA)-based anti-miR-141-3p oligonucleotide is present in the biocompatible nanoparticle in an amount of 0.01 to about 8 wt %, based on a total weight of the nanoparticle.

9. The pharmaceutically effective composition of claim 1, wherein the nanoparticles have an average particle sizes as determined by light scattering of 10 to 350 nanometers.

10. The pharmaceutically effective composition of claim 3, wherein the poly(lactic-co-glycolic acid) nanoparticles are derived from a combination of acid terminated poly(lactic-co-glycolic acid) and ester-terminated poly(lactic-co-glycolic acid).

11. A formulation comprising the pharmaceutically effective composition of claim 1.

12. The formulation of claim 11, where the formulation further comprises a carrier liquid.

13. The formulation of claim 12, where the carrier liquid comprises water, a biocompatible alcohol, saline, or a combination thereof.

14. The formulation of claim 13, where the biocompatible alcohol comprises ethanol, glycerol, propylene glycol, sorbitol, mannitol, polyethylene glycol, xylitol, erythritol, butylene glycol, or a combination thereof.

15. A method of using a pharmaceutical composition, the method comprising:
administering a pharmaceutically effective dose of the pharmaceutical composition to a patient;
wherein the pharmaceutical composition comprises at least one of:
a phosphorothioate (PS)-based anti-miR-141-3p oligonucleotide;
a peptide nucleic acid (PNA)-based anti-miR-141-3p oligonucleotide; and/or
a gamma-peptide nucleic acid (γ-PNA)-based anti-miR-141-3p oligonucleotide; where at least one of the foregoing oligonucleotides is encapsulated in a biocompatible nanoparticle; and
monitoring a symptom of the patient; and
modulating the pharmaceutically effective dose in response to observed results from the monitoring.

16. The method of claim 15, wherein the administering includes orally administering, intravenously administering, transdermally administering, topically administering and/or subcutaneously administering.

17. The method of claim 15, wherein the administering includes intravenously administering.

18. The method of claim 15, wherein the pharmaceutically effective dose comprises about 0.4 to about 4 microgram per kilogram of human weight.

19. A method of manufacturing a pharmaceutical composition, the method comprising:
dispersing a phosphorothioate (PS)-based anti-miR-141-3p oligonucleotide;
a peptide nucleic acid (PNA)-based anti-miR-141-3p oligonucleotide; and/or a gamma-peptide nucleic acid (γ-PNA)-based anti-miR-141-3p oligonucleotide in aqueous solution;
emulsified the aqueous solution containing the oligonucleotides into an organic solvent containing a nanoparticle precursor to form a water-in-oil (W/O) emulsion;
emulsifying the water-in-oil emulsion into a water-in-oil-in-water; and
drying the water-in-oil-in-water emulsion to form nanoparticles.

20. The method of claim 19, wherein the nanoparticle precursor is at least one of acid terminated poly(lactic-co-glycolic acid), ester-terminated poly(lactic-co-glycolic acid), or a combination thereof.


SEQUENCE LISTING (1 sequence)

SEQ ID NO: 1 - RNA, 24 nt, synthetic construct
  source 1..24: mol_type = other RNA
  misc_RNA 1..24: function = serine gamma-PNA
kccatctttaccagacagtgttak